(19) United States
(12) Plant Patent Application Publication
(10) Pub. No.: US 2016/0295764 P1
(43) Pub. Date: Oct. 6, 2016

(54) Abutilon plant named 'RutAbut2'

(50) ;

(71) Applicant: University of Georgia Research Foundation, Inc., Athens, GA (US)

(72) Inventors: John M. Ruter, Bishop, GA (US); Donglin Zhang, Athens, GA (US); Allan M. Armitage, Athens, GA (US); David A. Knauft, Watkinsville, GA (US); Suzzanne C. Tate, Athens, GA (US); Kelly Preslar, Signal Mountain, TN (US)

(73) Assignee: University of Georgia Research Foundation, Inc., Athens, GA (US)

(21) Appl. No.: 14/545,199

(22) Filed: Apr. 6, 2015

(51) Int. Cl. A01H 5/00

(52) U.S. Cl. PLT/226

(57) ABSTRACT

And Abutilon plant named ‘RutAbut2’ has prolific quantities of large yellow flowers, a compact and more prostrate growth habit, shorter internodes, and has an extended duration of flowering.

### STATEMENT REGARDING FEDERALLY-SPONSORED RESEARCH

This invention was made, in part, with U.S. Government support on behalf of U.S. Department of Agriculture, Hatch Act Grant. The U.S. Government has certain rights in this invention.

### GENUS AND SPECIES

Abutilon×hybridum.

### VARIETY DENOMINATION

The new Abutilon×hybridum claimed is of the cultivar denominated ‘RutAbut2’.

### BACKGROUND OF THE INVENTION

The present invention relates to a new and distinct cultivar of Abutilon×hybridium, hereinafter referred to by the varietal denomination ‘RutAbut2’.

Pedigree and history: ‘RutAbut2’ is a product of a cross made between Abutilon ‘Fairy Coral Red’ (unpatented, female parent) and Abutilon ‘Halo’ (unpatented, male parent) in 2007 under the direction of David Knauft and Suzzanne Tate. Cuttings were first taken in March of 2008 and were planted in Watkinsville, Ga. in May of 2008. Additional plants of the ‘RutAbut2’ variety, produced by cuttings, were transferred to a location in Bonsall, Calif. in June of 2012 for further evaluation. ‘RutAbut2’ has been evaluated through trials at locations in Athens, Ga. from 2009 to 2013; in Tifton , Ga. from 2009 to 2012, and Bonsall, Calif. from 2012 to 2013.

‘RutAbut2’ has been cold hardy outside in Tifton, Ga. in unprotected containers sitting above ground. Flowering occurs on ‘RutAbut2’ from mid-April until hard frost in Athens, Ga., whereas in contrast most selections of Abutilon stop flowering in heat of summer.

The Inventors would expect the cultivar to be used commercially as a perennial in USDA zones 8b-11. ‘RutAbut2’ can be used as a flowering annual in colder parts of the United States. Production is expected in the southeastern and southwestern United States. ‘RutAbut2’ should perform well in other countries with similar climates. ‘RutAbut2’ can be used in hanging baskets.

### SUMMARY OF THE INVENTION

The following represent the distinguishing characteristics of the new Abutilon cultivar named ‘RutAbut2’. In combination, these traits set ‘RutAbut2’ apart from all other existing varieties of Abutilon known to the Inventors. Asexual propagation by cuttings has proven that these characteristics are firmly fixed in succeeding asexually propagated generations.

1. Extended duration of flowering.

2. Improved, more compact habit.

3. Shorter internodes.

4. More prostrate growth.

5. Prolific.

6. Larger yellow flowers.

Plants of the new Abutilon can be compared to ‘Canary Bird’ (unpatented) in the industry. ‘RutAbut2’ is an improvement over the industry standard because it has increased flowering in comparison to ‘Canary Bird’.

Compared to its female parent ‘Fairy Coral Red’, ‘RutAbut2’ has larger flowers and foliage.

Compared to its male parent ‘Halo’, ‘RutAbut2’ has a less dark calyx and petals that are not as yellow.

TABLE 1
Flower sizes and color information for Abutilon varieties
listed below; growing in a trial garden in Athens,
Georgia (data were collected on Sep. 11, 2012)

| | Diameter (cm) | | Length (cm) | | |
|---|---|---|---|---|---|
| Clone | Mean | Range | Mean | Range | Note |
| ‘A08-0401’ | 3.4 | 2.9-3.7 | 3.8 | 3.5-4.0 | Bright red, stamens |
| | | | | | insert, heavy bloom. |
| ‘RutAbut2’ | 2.1 | 1.7-2.3 | 3.5 | 3.4-3.5 | Pure yellow, stamens |
| | | | | | exsert, only a few |
| | | | | | flowers |
| ‘RutAbut1’ | 2.9 | 2.2-4.0 | 3.2 | 2.6-3.5 | Yellow-orange with |
| | | | | | red throat, stamens |
| | | | | | exsert, heavy to |
| | | | | | medium bloom |
| ‘A08-2110’ | 5.7 | 4.8-6.3 | 3.7 | 3.4-4.1 | Pinkish red |
| | | | | | (watermelon red), |
| | | | | | stamens exsert, |
| | | | | | medium bloom |
| ‘A08-2112’ | 6.0 | 4.8-6.8 | 3.9 | 3.4-4.3 | Red, stamens exsert, |
| | | | | | heavy bloom |
| ‘A08-2114’ | 5.7 | 4.9-6.1 | 3.8 | 3.6-4.1 | Yellow with distinguished |
| | | | | | red venation, stamens |
| | | | | | exsert, heavy bloom |
| ‘A08-2121’ | 2.9 | 2.2-3.8 | 3.9 | 3.2-4.7 | Light pinkish red, stamens |
| | | | | | insert, only a few flowers |
| ‘A08-2125’ | 5.5 | 4.5-6.1 | 4.4 | 4.0-4.6 | Yellow with red marginal |
| | | | | | veins, stamens exsert, |
| | | | | | heavy bloom |
| ‘FCR’ | 1.3 | 1.0-1.7 | 3.4 | 3.0-3.7 | Deep red, stamens |
| | | | | | exsert, only 3 flowers |
| ‘A08-2131’ | 5.4 | 4.5-6.2 | 3.7 | 3.4-4.2 | Pinkish red, stamens |
| | | | | | exsert, heavy bloom |

The above varieties are all unpatented. The A08 prefix designated clones and ‘FCR’ designation are experimental variety designations that may be changed at a later time. ‘RutAbut1’ is to be the subject of a U.S. Plant Patent Application.

### BRIEF DESCRIPTION OF THE FIGURES

The accompanying colored photographic illustrations show the overall appearance and distinct characteristics of the new ‘RutAbut2’ cultivar. The colors in the photographs are as close as possible with the photographic and printing technology utilized.

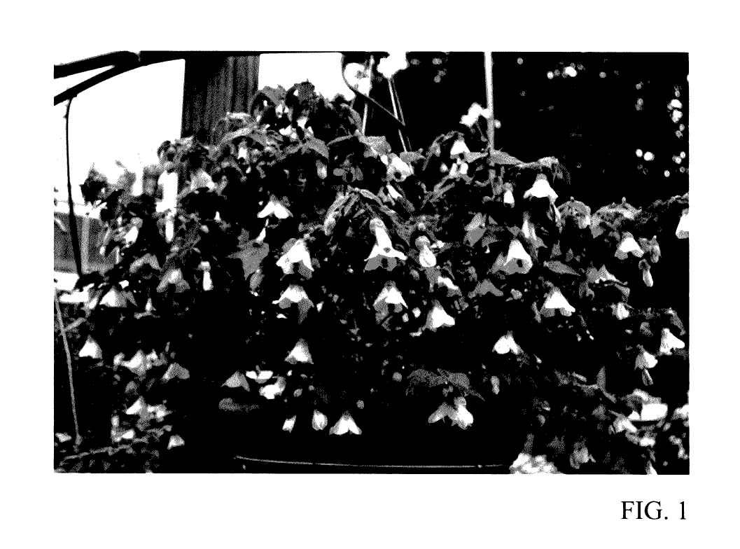

The photograph labeled FIG. 1 depicts ‘RutAbut2’.

### DETAILED BOTANICAL DESCRIPTION

The following is a detailed description of the Abutilon cultivar named ‘RutAbut2’. Data was collected in Tifton, Ga. from 12 month old plants grown from cuttings and growing outdoors in unprotected containers sitting above ground. ‘RutAbut2’ has not been tested under all possible conditions hence, phenotypic differences may be observed with variations in environmental conditions without any variance in genotype.

Throughout this specification, color names beginning with a small letter signify that the name of that color, as used in common speech, is aptly descriptive. Color names beginning with a capital letter designate values based upon the R.H.S. Colour Chart, 5^{th} edition published by The Royal Horticultural Society (R.H.S.), London, England.
Parentage: ‘RutAbut2’ is a seedling selection resulting from the deliberate controlled crossing of the following parents:
   Female parent.—‘Fairy Coral Red’.
   Male parent.—‘Halo’.
Vigor: Fairly vigorous.
Growth habit: Upright spreading.
Type: Perennial in zones where it is hardy.
Size:
   Height (cm).—94.
   Width (cm).—88×105 — (typical, measurements taken at narrowest and widest width).
Stem:
   Branching habit.—Freely.
   Stem length (cm) typical range.—10 to 40.
   Stem diameter (mm) typical.—2.
   Stem texture.—Smooth.
   Color.—Upper — Green Group 137A Lower — Green Group 143A.
   Pubescence.—Yes.
   Shape.—Round.
   Odor (of bruised stem).—None.
   Internode (length).—3cm.
Leaf:
   Upper color.—Young — Green Group 137A, 137B Grown up — Green Group 139A.
   Lower color.—Young — Green Group 137A, 137B Grown up — 139A.
   Mature size (L×W) (cm).—8.7×5.8.
   Apex.—acuminate.
   Base.—cordate.
   Margin.—serrated.
   Shape.—lobed.
   Number of lobes.—1 lobe per side.
   Pubescence.—Present on both upper and lower leaf surfaces.
   Arrangement on stem.—alternate.
   Venation.—netted.
   Vein color, upper surface.—Yellow-Green Group 146B.
   Vein color, lower surface.—Yellow-Green Group 147C.
   Texture.—papery.
Petiole:
   Length (cm).—2.5.
   Shape.—round.
   Color.—Upper — Green Group 137A Lower — Green Group 143A.
   Pubescence.—Yes.
   Diameter (mm).—1.
   Stipules.—none.
Flower:
   Inflorescence.—Number of individual flowers per stem — generally one flower per leaf node.
   Individual flower.—Size (L×W), typical (cm): 3 to 4×4.5 Axillary or terminal — axillary Symmetry — actinomorphic Fragrance: none Petal Number per flower: five Petal — Size (L×W), typical, (cm) — 4.1×2.6 Shape — spatular Apex — round Base — attenuate Margin — entire Color at peak of bloom — Upper: Yellow-Orange group 15C Center: Yellow-Orange group 15C Petals: Not fused Bud color: Greyed-Orange group 173A Bud shape: Ovoid Bud size (typical) Length (cm): 1.5 Width (cm at widest point): 1 Bud apex: Cuspidate Bud surface: Pubescent Pedicels — Color — Green Pubescence — Yes Length (cm) — 6.6 Sepals — Number — 5 calyx Size (cm) — 1.6 Shape — base connects, top divides Pubescence — Yes Color — Greyed-Red 179C Stamens — Number — approximately 120 Size — 0.2 cm in length and 0.5 mm in diameter Color — red Pollen Color — Yellow-Orange group 17C Pubescence — none Anther color: Brown group 200D Anther shape: Elliptical Pollen quantity: Moderately heavy Pollen color: Yellow group 12A Pistils — Size of style — 3.3cm long Color of style — Greyed-Purple 184D Color of stigma — Greyed-Purple 185A.
Seed: None observed
Hardiness: Proven hardy in USDA zones 8a and 10a where it has been grown and evaluated as of this time. Full range of USDA zones where the plant is hardy has not yet been determined.
Disease resistance: No unusual disease resistance or susceptibility has been observed to date.

What is claimed is:

1. A new and distinct cultivar of the Abutilon×hybridum plant named ‘RutAbut2’ substantially as illustrated and described herein.
